(19) United States
(12) Patent Application Publication
(10) Pub. No.: US 2011/0184440 A1
(43) Pub. Date: Jul. 28, 2011

(54) MAGNETICALLY MANIPULABLE SURGICAL MESH AND APPARATUS FOR THE MANIPULATION THEREOF

(76) Inventor: Pierre F. Saldinger, Weston, CT (US)

(21) Appl. No.: 13/013,861

(22) Filed: Jan. 26, 2011

Related U.S. Application Data
(60) Provisional application No. 61/298,241, filed on Jan. 26, 2010.

(51) Int. Cl. A61B 17/00

(52) U.S. Cl. 606/151

(57) ABSTRACT

A surgical mesh for reinforcing a muscle wall is disclosed. The mesh is made from a flexible material such that the mesh can be rolled to allow for insertion into a body via an incision. A magnetic material is attached to the mesh. The mesh can be manipulated inside of the body by applying a magnetic force outside of the body. In some embodiments, the magnetic material is disposed across the entire mesh to allow the mesh to be sized for a unique surgical case, while still providing sufficient magnetic material to allow for manipulation of the mesh. In some embodiments, an apparatus is used to position external magnets outside of the body for manipulating the mesh inside of the body.

### CROSS-REFERENCE TO RELATED APPLICATIONS

The present application claims the benefit under 35 U.S.C. §119 (e) of the Provisional Patent Application Ser. No. 61/298,241 filed Jan. 26, 2010, the disclosure of which is incorporated by reference in its entirety.

### FIELD OF THE INVENTION

The present invention relates to surgical mesh for use in a body. More specifically, the present invention relates to a magnetically manipulable surgical mesh, an apparatus for the manipulation thereof, and methods for producing and deploying a magnetically manipulable surgical mesh.

### BACKGROUND OF THE INVENTION

Hernias erupt when a weakened abdominal muscle tears open, permitting the organs inside to push through. Typically, a hernia will pouch out, looking like a balloon beneath the skin. Two areas are especially vulnerable.

Inguinal hernias appear at the point where the leg joins the abdomen. Men feel this type of hernia as a lump in the scrotum. Umbilical hernias crop up at the navel. Surgical scars present yet another opportunity for hernias—in this case called incisional hernias.

A hernia repair is known medically as a herniorrhaphy. Hernia can be repaired laparoscopically and generally involve the placement and attachment of a manmade mesh material to the inside of a repaired torn muscle wall to further strengthen it. The mesh is introduced into a patient's body by being rolled-up and fed through a tube into the body. Once free of the tube the mesh must be unrolled and manipulated into position. The mesh is then held in position and attached to the muscle wall. One difficulty that occurs is that it can be difficult to position the mesh using laparoscopic instruments.

### SUMMARY OF THE INVENTION

The present inventions relates to a surgical mesh. The mesh is made from a flexible material that can be rolled to allow for insertion of the mesh into a body of a patient via an incision in the body, for example through a trocar commonly used in laparoscopic surgery. A magnetic material is attached to the mesh. The mesh can be manipulated inside of the body by applying a magnetic force outside of the body to affect the magnetic material attached to the mesh.

In some embodiments of the present invention, the magnetic material comprises a plurality of discrete pieces arranged on a periphery of the mesh. In yet other embodiments, the discrete pieces are disposed across the mesh.

The magnetic material can comprise a strip. The strip may extend along a portion of the periphery of the mesh, or extend along substantially all of the periphery of the mesh. In some embodiments, a plurality of strips can extend outward from a first area on the mesh forming a hub and spoke pattern. The first area can be located substantially in the center of the mesh. The strips can comprise one or more of a magnetic thread woven into the mesh, a magnetic coating applied to the mesh, and a magnetic tape adhered to the mesh. In some embodiments of the present invention an apparatus is provided for positioning one or more magnets outside of the body to manipulate a mesh.

### BRIEF DESCRIPTION OF THE DRAWINGS

FIG. 1 is a top view of a piece of surgical mesh having magnetic material positioned at discrete locations along the mesh periphery.

FIG. 2 is a top view of a piece of surgical mesh wherein the magnetic material extends along the periphery of the mesh.

FIG. 3 is a top view of a piece of surgical mesh wherein the magnetic material extends throughout the mesh in a plurality of threads.

FIG. 4 is a cross-sectional view of a piece of surgical mesh wherein the surgical mesh is manipulated inside a body using a plurality of external magnets from outside of body.

FIG. 5 is a top view of an apparatus used from outside the body to position a surgical mesh having magnetic material disposed therein.

### DETAILED DESCRIPTION OF THE INVENTION

As shown in FIG. 1 a piece of surgical mesh material is generally designated by the reference numeral 10. The surgical mesh 10 can be made from any suitable polymer, textile, metallic weave, or other material and combinations, such as but not limited to polypropylene. A plurality of discrete pieces of magnetic material 12 are attached to the mesh and are positioned around a periphery defined by the mesh 10.

The magnetic material 12 can be any suitable material or configuration, such as, but not limited to magnets, magnetic metals, composite materials containing magnetic material, polymeric beads, discs or other shaped elements coated or impregnated with magnetic material, and combinations thereof. The magnetic material 12 is attached to the mesh and, as will be explained in detail below, is manipulated while within a body cavity using magnets outside of the body. While the discrete pieces of magnetic material 12 are shown and described as being located on the periphery of the mesh 10, the present invention is not limited in this regard as the pieces of magnetic material 12 can be located anywhere on the mesh without departing from the broader aspects of the present invention.

Referring to FIG. 2, a piece of surgical mesh, generally designated by the reference number 20, includes a strip of magnetic material 22 extending around the periphery of the mesh 20. The strip of magnetic material 22 is fixed relative to the mesh 20. The strip of magnetic material 22 can comprise, but is not limited to, magnetic thread woven into the mesh, a magnetic coating, tape adhered to the mesh, or combinations thereof.

The surgical mesh 20 can be magnetically manipulated when in a body cavity, in the same manner as described above for the surgical mesh 10, and described in greater detail below. While the surgical mesh 20 has been described as having a strip of magnetic material 22 extending along its periphery, the present invention is not limited in this regard as the strip of magnetic material 22 could extend intermittently around the periphery or along two sides of the periphery without departing from the broader aspects of the invention.

One potential issue regarding the above-described surgical mesh 10 and 20 occurs if the mesh must be cut to a particular size or shape to accommodate a particular surgical case. If this proves necessary, then a portion of the magnetic material may be cut off with a discarded piece of mesh, making the mesh difficult to manipulate and deploy using external magnets.

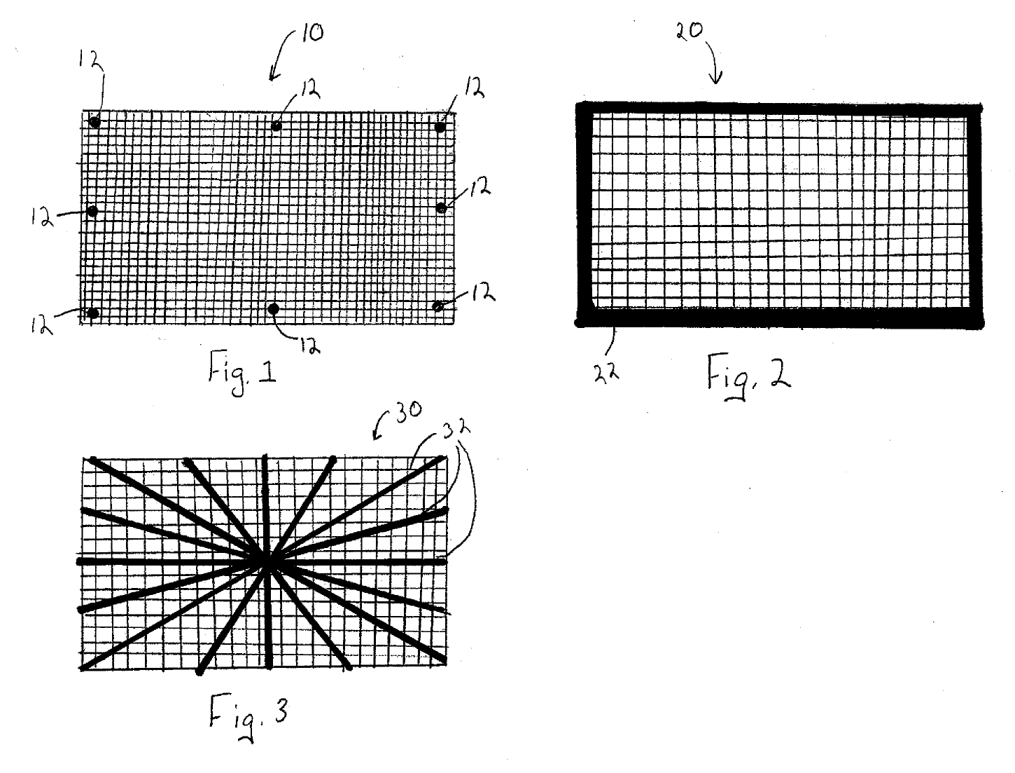

As shown in FIG. 3, the surgical mesh generally designated by the reference number 30 includes a plurality of strips of magnetic material 32 in a spider-web type pattern. The spider-web pattern, also referred to as a hub and spoke pattern, allows the surgical mesh 30 to be cut to a particular shape required for the surgical case, yet still have sufficient magnetic material located thereon to allow the entire piece of surgical mesh to be manipulated into place. The strips of magnetic material 32 can be woven into the surgical mesh 30, adhered to an outer surface of the surgical mesh, or be applied as a coating onto the surgical mesh 30. If woven into the surgical mesh 30, the magnetic material 32 can take the form of a magnetic thread. The magnetic thread can be metallic, polymeric coated with or impregnated with a magnetic material, combinations thereof, and the like.

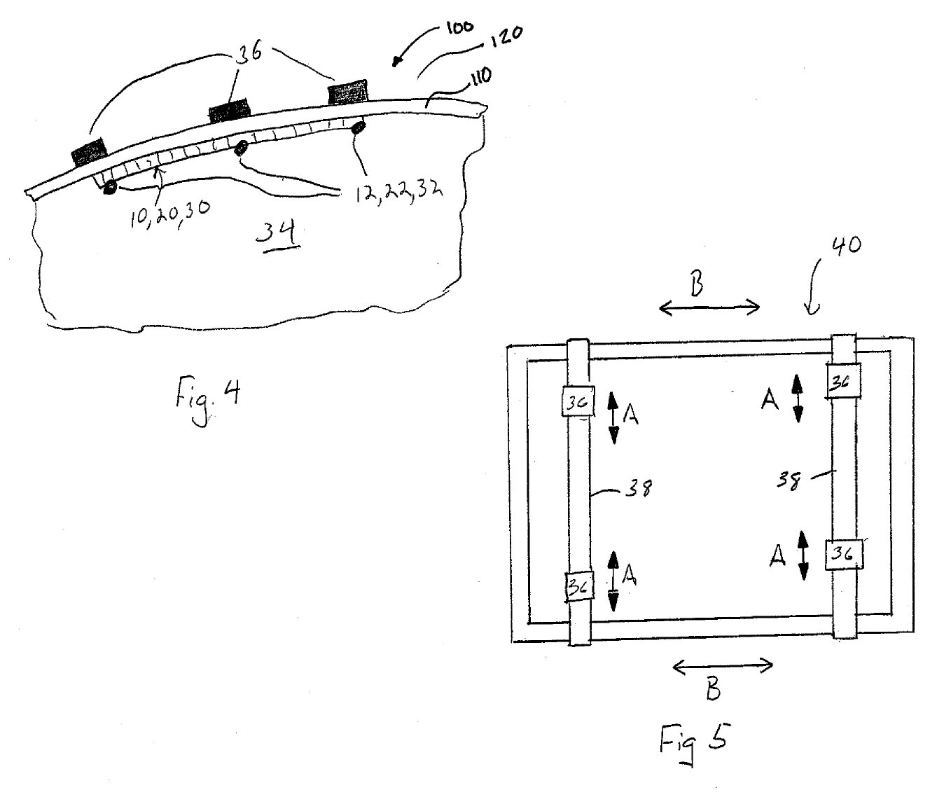

As shown in FIG. 4, during a surgical procedure where surgical mesh 10, 20, or will be positioned within a body 34, the mesh can be manipulated using magnets 36 manipulated outside of the body. The magnets 36 attract the magnetic material 12, 22, 32 thereby allowing the surgical mesh 10, 20, 30 to be moved, stretched and otherwise desirably positioned within the body 34. Once positioned, the surgical mesh 10, 20, 30 can be surgically attached to the body.

The magnets 36 can be individual magnets that are manipulated by hand. The magnets can be of the permanent or electromagnetic type. The magnets 36 can also be movably mounted to a frame 40 as shown in FIG. 5. In the illustrated embodiment, an apparatus is provided for use outside the body for precisely guiding the external magnets 36 for deploying and/or positioning the surgical mesh inside of the body. The apparatus shown in FIG. 5 includes two rails 38. The external magnets 36 are slideably disposed on the rails and can move back and forth along the axis “A” along the rails 38. The rails 38 can also move back and forth as indicated by the arrows labeled “B” along the frame 40. The apparatus 40 can be placed over the surgical site to precisely position the surgical mesh. In some embodiments, the apparatus is supported by a surgical ceiling mount. In other embodiments of the present invention, the apparatus is placed on a surgical table above the patient. In yet other embodiments of the present invention, the apparatus is rested on the patient, or held above or proximate to the patient with a stand.

In some embodiments of the present invention the position of the external magnets 36 on the apparatus is remotely controlled. In such cases, it is possible to precisely control the position of the external magnets. Remote control of the external magnets may be achieved in a number of different manners. For example, the position of the slidably disposed magnets on the rail is controlled by an electric motor fixed to the rail. Similarly, the position of the rails along axis “B” can also be control by one or more electric motors. The electric motors can be operated by a central control box. In some embodiments of the present invention, a control system for positioning the external magnets is incorporated into a more general surgical control system, for example as system that controls surgical devices, including audio/video, in the operating room. The user can remotely control the external magnets via a user interface in communication with the control system, for example a wireless tablet or a touch screen. In some embodiments, the surgeon can view of the mesh inside the body using one or more endoscopic camera inserted into the body. The user can input commands on the touch screen to position the mesh. Based on the commands, the control system controls the location of the external magnets in accordance with the received instructions.

Although the present invention has been disclosed and described with reference to certain embodiments thereof, it should be noted that other variations and modifications may be made, and it is intended that the following claims cover the variations and modifications within the true scope of the invention.

What is claimed is:

1. A surgical device for deployment inside of a body, the device comprising:
a flexible surgical mesh that can be rolled to allow for insertion of the mesh into a body though an incision in the body; and
a magnetic material attached to the mesh,
wherein the mesh is manipulable inside the body by applying a magnetic force from outside the body.

2. The surgical device of claim 1, wherein the magnetic material comprises a plurality of discrete pieces, the plurality of discrete pieces arranged on a periphery of the mesh.

3. The surgical device of claim 2, wherein the discrete pieces of magnetic material are disposed across the mesh.

4. The surgical device of claim 1, wherein the magnetic material comprises at least one strip.

5. The surgical device of claim 4, wherein the strip extends along at least a portion of the periphery of the mesh.

6. The surgical device of claim 5, wherein the strip extends along substantially the entire periphery of the mesh.

7. The surgical device of claim 3, wherein the strip comprises one or more of a magnetic thread woven into the mesh, a magnetic coating applied to the mesh, and a magnetic tape adhered to the mesh.

8. The surgical device of claim 4, wherein the magnetic material comprises a plurality of magnetic strips, a proximal end defined by each of the strips is attached to the mesh at a first area, and wherein each of the strips extends outwardly from the first area.

9. The surgical device of claim 8, wherein the first area is located substantially in a center of the mesh.

10. A system for reinforcing a muscle wall inside of a body, the system comprising:
a flexible surgical mesh that can be rolled to allow for insertion of the mesh into a body though an incision in the body, the mesh having a magnetic material attached thereto; and
an apparatus for manipulating one or more magnets outside of the body for positioning the mesh inside the body, the apparatus having one or more rails extending along a first axis and one or more magnets slideably disposed on each of the one or more rails;
wherein the one or more rails are movable along a second axis perpendicular to and coplanar with the first axis,
wherein the mesh is manipulable inside the body by applying a magnetic force from outside the body by moving one or more of the magnets disposed on the apparatus.

11. The system of claim 10, wherein the apparatus is fixed relative to the body by one or more of a ceiling mount, floor stand, and surgical table.

12. The system of claim 11, wherein the one or more magnets can be positioned remotely using a control system configured to position the external magnets.

13. A method of securing a tissue inside a body, the method comprising the steps of:
providing a surgical mesh, the mesh having a magnetic material attached thereto;
rolling the mesh into a tube-like shape;
inserting the rolled mesh via an incision in the body into a cavity in the body;
positioning the mesh inside the body by apply a magnetic force from outside the body; and
attaching the mesh to tissue inside the body.

14. The method of claim 13, further comprising the steps of:
cutting the mesh to a specific size to accommodate a unique surgical case prior to inserting the mesh into the body.

15. The method of claim 14, wherein the magnetic material comprises a plurality of magnetic strips, a proximal end defined by each of the strips is attached to the mesh at a first area, and wherein each of the strips extends outwardly from the first area.

16. The method of claim 15, wherein the first area is located substantially in a center of the mesh.

17. The method of claim 14, further comprising the step of:
providing an apparatus for manipulating one or more magnets outside of the body for positioning the mesh inside the body, the apparatus having one or more rails extending along a first axis and one or more magnets slideably disposed on each of the one or more rails;
wherein the one or more rails are movable along a second axis perpendicular to and coplanar with the first axis;
wherein the mesh is manipulable inside the body by applying a magnetic force from outside the body by moving one or more of the magnets disposed on the apparatus.
